(19) United States
(12) Patent Application Publication
(10) Pub. No.: US 2025/0352461 A1
(43) Pub. Date: Nov. 20, 2025

(54) BEVERAGE COMPOSITIONS FOR SUPPORT OF HAIR HEALTH

(71) Applicant: REMI INVESTMENT HOLDINGS, LLC, Henderson, NV (US)

(72) Inventor: SHAHIN SHANE MALEK, Henderson, NV (US)

(21) Appl. No.: 19/212,037

(22) Filed: May 19, 2025

Related U.S. Application Data
(60) Provisional application No. 63/649,363, filed on May 18, 2024.
(60) Provisional application No. 63/664,723, filed on Jun. 27, 2024.

(51) Int. Cl. A61K 8/98; A61K 8/04; A61K 8/27; A61K 8/44; A61K 8/67; A61K 8/9728; A61K 8/9789; A61Q 5/00

(52) U.S. Cl. CPC A61K 8/986; A61K 8/04; A61K 8/27; A61K 8/44; A61K 8/673; A61K 8/675; A61K 8/676; A61K 8/9728; A61K 8/9789; A61Q 5/00; A61K 2800/92

(57) ABSTRACT

Orally consumable beverage compositions for support of head hair health are described, which may be formulated as dairy milk beverage compositions fortified with hair benefit ingredients, or as non-dairy milk substitute beverage compositions fortified with head hair benefit ingredients. The head hair benefit ingredients may include (i) trace elements, and/or oxides and/or salts and/or derivatives thereof; (ii) vitamins and derivatives thereof; (iii) proteins; (iv) amino acids; and (v) plant extracts.

### CROSS-REFERENCE TO RELATED APPLICATIONS

The benefit under 35 USC § 119 of U.S. Provisional Patent Application 63/649,363 filed May 18, 2024 for BEVERAGE COMPOSITIONS FOR SUPPORT OF HAIR HEALTH, and the benefit under 35 USC § 119 of U.S. Provisional Patent Application 63/664,723 filed Jun. 27, 2024 for BEVERAGE COMPOSITIONS FOR SUPPORT OF HAIR HEALTH, are hereby claimed. The disclosures of U.S. Provisional Patent Application 63/649,363 and U.S. Provisional Patent Application 63/664,723 are hereby incorporated herein by reference, in their respective entireties, for all purposes.

### FIELD

The present disclosure generally relates to beverage compositions for support of hair health, and more specifically to dairy milk beverage compositions fortified with hair benefit ingredients, and to non-dairy milk substitute beverage compositions fortified with hair benefit ingredients.

### DESCRIPTION OF THE RELATED ART

A large number of topically applied hair growth compositions are known in the art, utilizing a variety of active ingredients for promoting hair growth of persons who suffer from hair loss. There also exist many different types of shampoos, conditioners, and hair rinses that are formulated for topical application to the hair to support hair health and beauty.

Various disadvantages of topically applied hair benefit compositions include adverse dermatological and systemic side effects, lengthy times and inconvenient application requirements for topical contacting with the hair benefit composition to achieve the desired enhancement effect, and the presence in many products of ingredients that are not environmentally benign.

### SUMMARY

The present disclosure relates broadly to beverage compositions for human oral consumption for support of hair health, including dairy milk beverage compositions fortified with hair benefit ingredients and nutrients, and non-dairy milk substitute beverage compositions fortified with hair benefit ingredients and nutrients.

The present disclosure in one aspect relates to a beverage composition for oral consumption for support of head hair health, comprising (i) dairy milk, or non-dairy milk substitute, and (ii) one or more head hair benefit ingredient(s).

In another aspect, the present disclosure relates to a beverage composition for oral consumption for support of hair health, comprising any one or more of soy milk, biotin, zinc, thiamine, reishi mushroom extract, lion's mane mushroom extract, ashwagandha root extract, foti root extract, horsetail extract, Pygeum africanum, plant sterols (which may be or comprise sterols and stanols present in the plant extracts of the beverage composition, such as foti root extract, horsetail extract, etc., or alternatively may be separately and additionally added to the beverage composition, such as beta-sitosterol, stanol ester, campesterol, avenasterol, isofucosterol, sterolin, brassicasterol, sterol ester, stigmasterol, etc.), protein(s) (such as branched chain proteins), vitamins, minerals, and amino acid(s), e.g., branched chain amino acids (BCAA) such as leucine, isoleucine, and valine, which may be in a BCAA blend, such as a BCAA 2:1:1 blend of L-leucine: L-isoleucine, and L-valine, or other suitable blend formulation.

In another aspect, the disclosure relates to a dairy milk beverage composition for support of head hair health, comprising (i) dairy milk and (ii) one or more head hair benefit ingredient(s).

In a further aspect, the disclosure relates to a non-dairy milk substitute beverage composition for support of head hair health, comprising (i) non-dairy milk substitute and (ii) one or more head hair benefit ingredient(s).

Another aspect of the disclosure relates to a method of mediating head hair health of a human subject in need thereof, comprising administering to the human subject a beverage composition of the present disclosure.

Other aspects, features and embodiments of the disclosure will be more fully apparent from the ensuing description and appended claims.

### DETAILED DESCRIPTION

The present disclosure generally relates to orally consumable beverage compositions for support of hair health, and more specifically to dairy milk beverage compositions fortified with hair benefit ingredients, and to non-dairy milk substitute beverage compositions fortified with hair benefit ingredients.

As used herein and in the appended claims, the singular forms “a”, “an”, and “the” include plural referents unless the context clearly dictates otherwise.

In all compositions of the present disclosure, all ingredients are present in respective physiologically acceptable amounts for human oral consumption.

In various embodiments, dairy milk beverage compositions comprise (i) dairy milk and (ii) one or more head hair benefit ingredient(s). The dairy milk may be of any suitable type, for example, cow milk, goat milk, sheep milk, zebu milk, buffalo milk, etc., and may be of any suitable fat content or may be fat-free, and may be lactose-free or lactose-containing.

The non-dairy milk substitute beverage compositions of the present disclosure may include any of a wide variety of plant-based non-dairy milk substitutes, including for example soy milk, almond milk, oat milk, coconut milk, rice milk, cashew milk, macadamia milk, hemp milk, quinoa milk, and other plant and nut milks.

In various embodiments, the dairy milk beverage compositions and the non-dairy milk substitute beverage compositions of the present disclosure may contain hair benefit ingredients such as any one or more of the following ingredients:
(i) trace element(s) such as zinc, calcium, selenium (e.g., selenomethionine), manganese, potassium, sodium, and iron, among others, and/or oxides and/or salts and/or derivatives (e.g., citrate, chloride, sodium, and sulfate salts, and benzoates and gluconates) thereof, such as magnesium citrate, potassium sorbate, potassium iodine (potassium iodide), sodium benzoate, sodium gluconate, zinc citrate and sodium chloride (e.g., sea salt);
(ii) vitamin(s) such as: vitamin A; vitamin B, e.g., riboflavin (vitamin B1), biotin (vitamin B7), folate (vitamin B9), thiamine (vitamin B1), niacin and/or niacinamide (vitamin B3), pantothenic acid (vitamin B5), pyridoxine (vitamin B6), and cobalamins (vitamin B12), etc.; vitamin C (ascorbic acid); vitamin D; vitamin E; and vitamin K; and derivatives thereof, such as for example thiamine HCl;
(iii) protein(s) such as soy protein, egg white protein, pea protein, whey protein, casein protein, and hemp protein, collagen, shark cartilage, and other plant and animal proteins, preferably branched chain proteins;
(iv) amino acid(s) such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, which may for example in various embodiments be provided in the form of blends of branched chain amino acids, such as L-leucine, L-isoleucine, and L-valine, in a weight ratio of 2:1:1 of L-leucine: L-isoleucine: L-valine (BCAA 2:1:1), taurine, and L-theanine; and
(v) plant extract(s) such as any of the extracts of amla, ashwagandha, reishi mushroom, lion's mane mushroom, foti root (Polygonum multiflorum), holy basil (Ocimum tenuiflorum), horsetail (Equisetum arvense), turmeric root, Pygeum africanum, mountain ash (Sorbus aucupario L. Rosaceae) berries, saw palmetto (Serenoa repens) fruit, raspberry juice concentrate, plant sterols and/or stannols (e.g., beta-sitosterol, stanol ester, campesterol, avenasterol, isofucosterol, sterolin, brassicasterol, sterol ester, stigmasterol), natural flavors (cocoa, vanilla, strawberry, lemon, lime, kiwi, banana), stevia extract, soy, vegetable glycerine, and citric acid, and the dairy milk beverage compositions and non-dairy milk substitute beverage compositions of the present disclosure may include any other hair benefit ingredient(s) that are physiologically acceptable for human oral consumption and that in the composition function to support head hair health, e.g., to promote head hair growth and healthy head hair characteristics. Trace metal elements such as zinc and selenium have been found to be particularly advantageous in various embodiments of the beverage compositions.

It will be recognized that hair benefit ingredients employed in the beverage compositions of the present disclosure may be in derivatized forms. For example, zinc when present may be in elemental form, or may be in an oxide compound, or may be in a salt compound (as zinc chloride, or other form), or may be in another compound, chelate, or complex containing zinc. Likewise, any of the proteins, amino acids, and/or plant extracts may be in derivatized forms in the beverage composition. For example, thiamine (thiamin) may be in present in the beverage composition as thiamine per se, or in a derivatized form such as thiamine hydrochloride (thiamine HCl).

The beverage compositions of the present disclosure may be formulated to contain a substantial protein content, including proteins as well as amino acids, which may for example the present in a total protein/amino acid content of up to 25 grams in a packaged beverage composition of 6-16 fluid ounces, e.g., in a range of from 1 to 25 grams of total protein/amino acid components, or in a range of from 5 to 20 grams of total protein/amino acid components, or in other suitable range for such fluid volume of the beverage composition.

In another aspect, the present disclosure relates to a beverage composition for oral consumption for support of hair health, comprising for example any of soy milk, biotin, zinc, thiamine, reishi mushroom extract, lion's mane mushroom extract, ashwagandha root extract, foti root extract, horsetail extract, Pygeum africanum, plant sterols (which may be or comprise sterols and stanols present in the plant extracts of the beverage composition, such as foti root extract, horsetail extract, etc., or alternatively may be separately and additionally added to the beverage composition, such as beta-sitosterol, stanol ester, campesterol, avenasterol, isofucosterol, sterolin, brassicasterol, sterol ester, stigmasterol, etc.), protein(s) (such as branched chain proteins), and amino acid(s), e.g., branched chain amino acids (BCAA) such as leucine, isoleucine, and valine, which may be in a BCAA blend, such as a BCAA 2:1:1 blend of L-leucine: L-isoleucine, and L-valine, or other suitable blend formulation.

In various embodiments, the dairy milk beverage compositions and non-dairy milk substitute beverage compositions of the present disclosure may comprise any one or more of the following hair benefit ingredients: biotin, zinc, thiamine, reishi mushroom extract, lion's mane mushroom extract, ashwagandha extract, foti root extract, plant sterols, horsetail extract, Pygeum africanum, protein, and BCAA 2:1:1.

In various embodiments, the dairy milk beverage compositions and non-dairy milk substitute beverage compositions of the present disclosure may comprise any one or more of the following ingredients: purified water, organic soy, plant protein blend, cocoa (or vanilla or kiwi or banana or strawberry or other natural flavor), magnesium citrate, potassium sorbate, sodium benzoate, sodium gluconate, sea salt, stevia leaf extract, ascorbic acid, zinc citrate, BCAA 2:1:1, niacinamide, biotin, lion's mane extract, reishi mushroom extract, ashwagandha root extract, foti root extract, horsetail extract, saw palmetto fruit extract, potassium iodine, selenomethionine, and thiamin HCl.

In various embodiments, the dairy milk beverage compositions and non-dairy milk substitute beverage compositions of the present disclosure may comprise any one or more of the following ingredients: riboflavin, biotin, vitamin D, zinc, selenium, beta-sitosterol, campesterol, stigmasterol, saw palmetto extract, horsetail extract, green tea extract, linolenic acid, turmeric extract, bamboo extract, Pygeum african extract, and fo-ti root extract.

In one specific embodiment, the beverage composition comprises a beverage composition for oral consumption for support of hair health, comprising soy milk, biotin, zinc, thiamine, reishi mushroom extract, lion's mane mushroom extract, ashwagandha root extract, foti root extract, plant sterols, horsetail extract, Pygeum africanum, protein, and BCAA 2:1:1.

In another specific embodiment, the beverage composition comprises dairy milk or non-dairy milk substitute; zinc; thiamine; lion's mane extract; saw palmetto extract; ashwagandha extract; bamboo extract; reishi mushroom extract; biotin; vitamin C; vitamin B-3; L-valine; L-leucine; L-isoleucine; Pygeum africanum; and horsetail extract.

The beverage compositions of the present disclosure in addition to supporting head hair health and promoting growth of head hair have also been found to be beneficial to support health of skin and nails.

The beverage compositions of the present disclosure may be provided in a suitable packaged form, comprising packaging containing the beverage composition, such as for example containing a single serving amount of the beverage composition, e.g., as a bottled or canned volume of 6 to 12 fluid ounces, or more, or as a multiple servings amount of the beverage composition, e.g., as a bottled or canned volume of 16 to 32 fluid ounces, or in other suitable volumes and packaged forms.

The present disclosure in a method aspect relates to a method of mediating head hair health of a human subject in need thereof, such method comprising administering to the human subject a beverage composition of the present disclosure, for oral consumption thereof.

Beverage compositions of the present disclosure can mediate enhanced growth of head hair in human subjects consuming such beverage compositions for sustained periods of 4-6 weeks, as compared to human subjects not consuming the beverage compositions but otherwise having substantially similar dietary, sleep, and exercise activity.

While the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

What is claimed is:

1. A beverage composition for oral consumption for support of hair health, comprising (i) dairy milk, or non-dairy milk substitute, and (ii) one or more head hair benefit ingredient(s).

2. The beverage composition of claim 1, comprising dairy milk.

3. The beverage composition of claim 2, wherein the dairy milk comprises any one or more of cow milk, goat milk, sheep milk, zebu milk, and buffalo milk.

4. The beverage composition of claim 1, comprising non-dairy milk substitute.

5. The beverage composition of claim 4, wherein the non-dairy milk substitute comprises a plant milk.

6. The beverage composition of claim 4, wherein the non-dairy milk substitute comprises a nut milk.

7. The beverage composition of claim 4, wherein the non-dairy milk substitute comprises one or more of soy milk, almond milk, oat milk, coconut milk, rice milk, cashew milk, macadamia milk, hemp milk, and quinoa milk.

8. The beverage composition of claim 4, wherein the non-dairy milk substitute comprises soy milk.

9. The beverage composition of claim 1, wherein the one or more head hair benefit ingredients are selected from the group consisting of:
(i) trace elements, and/or oxides and/or salts and/or derivatives thereof;
(ii) vitamins and derivatives thereof;
(iii) proteins;
(iv) amino acids; and
(v) plant extracts.

10. The beverage composition of claim 9, wherein the trace elements and/or oxides and/or salts thereof comprise zinc, calcium, selenium, selenomethionine, manganese, potassium, sodium, and iron.

11. The beverage composition of claim 9, wherein the salts and derivatives comprise magnesium citrate, potassium sorbate, potassium iodine, sodium benzoate, sodium gluconate, zinc citrate and sodium chloride.

12. The beverage composition of claim 9, wherein the vitamins and derivatives comprise: vitamin A, vitamin B, riboflavin (vitamin B1), biotin (vitamin B7), folate (vitamin B9), thiamine (vitamin B1), niacin and/or niacinamide (vitamin B3), pantothenic acid (vitamin B5), pyridoxine (vitamin B6), cobalamins (vitamin B12); vitamin C (ascorbic acid), vitamin D, vitamin E, vitamin K, and thiamine HCl.

13. The beverage composition of claim 9, wherein the proteins comprise soy protein, egg white protein, whey protein, casein protein, pea protein, hemp protein, collagen, and shark cartilage.

14. The beverage composition of claim 9, wherein the amino acids comprise alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

15. The beverage composition of claim 9, wherein the amino acids comprise a blend of L-leucine, L-isoleucine, and L-valine, in a weight ratio of 2:1:1 of L-leucine: L-isoleucine: L-valine (BCAA 2:1:1).

16. The beverage composition of claim 9, wherein the plant extracts comprise extracts of amla, ashwagandha, reishi mushroom, lion's mane mushroom, foti root (Polygonum multiflorum), holy basil (Ocimum tenuiflorum), horsetail (Equisetum arvense), turmeric root, Pygeum africanum, mountain ash (Sorbus aucupario L. Rosaceae) berries, saw palmetto (Serenoa repens) fruit, raspberry juice concentrate, beta-sitosterol, campesterol, stigmasterol, natural flavors, stevia extract, soy, vegetable glycerine, and citric acid.

17. The beverage composition of claim 1, comprising soy milk, biotin, zinc, thiamine, reishi mushroom extract, lion's mane mushroom extract, ashwagandha root extract, foti root extract, plant sterols, horsetail extract, Pygeum africanum, protein, and BCAA 2:1:1.

18. The beverage composition of claim 17, comprising one or more selected from the group consisting of plant proteins, cocoa, vanilla, strawberry, kiwi, banana, magnesium citrate, potassium sorbate, sodium benzoate, sodium gluconate, sea salt, stevia leaf extract, ascorbic acid, zinc citrate, niacinamide, saw palmetto fruit extract, potassium iodine, selenomethionine, and thiamine HCl.

19. A method of mediating head hair health of a human subject in need thereof, comprising administering to the human subject a beverage composition according to claim 1.

20. A dairy milk beverage composition for support of head hair health, comprising: dairy milk or non-dairy milk substitute; zinc; thiamine; lion's mane extract; saw palmetto extract; ashwagandha extract; bamboo extract; reishi mushroom extract; biotin; vitamin C; vitamin B-3; L-valine; L-leucine; L-isoleucine; Pygeum africanum; and horsetail extract.
